(19) United States
(12) Patent Application Publication
(10) Pub. No.: US 2012/0046525 A1
(43) Pub. Date: Feb. 23, 2012

(54) LAPAROSCOPIC SURGICAL RETRACTION DEVICE

(75) Inventors: Barry Russell, Kildare (IE); Ronan Keating, Galway (IE); Gerard Rabbitte, Galway (IE)

(73) Assignee: NEOSURGICAL LIMITED, Galway (IE)

(21) Appl. No.: 13/202,724

(22) PCT Filed: Feb. 22, 2010
(86) PCT No.: PCT/EP2010/052216

§ 371 Date: Nov. 2, 2011

(30) Foreign Application Priority Data
Feb. 23, 2009 (GB) 0903001.6

(51) Int. Cl. A61B 1/32

(52) U.S. Cl. 600/204

(57) ABSTRACT

A laparoscopic surgical retraction device is described. The device has an insertion configuration and an operational configuration. In the insertion configuration the device is collapsible such that the dimensions of the device can be reduced so as to allow the complete insertion of the device through a trocar or cannula into the internal abdominal cavity wherein it may be expanded to adopt the operational configuration.

### CROSS-REFERENCE TO RELATED APPLICATIONS

This application is a U.S. national stage application filed under 35 U.S.C. §371 of International Patent Application PCT/EP2010/052216, accorded an international filing date of Feb. 22, 2010, which claims benefit under 35 U.S.C. 119(e) to U.S. provisional patent application Ser. No. 61/154,466, filed Feb. 23, 2009, and incorporates by reference the contents of these applications in their entirety.

### FIELD OF THE INVENTION

The present invention relates to laparoscopic surgery. The invention more particularly relates to a retraction device for use in laparoscopic surgery. The invention also relates to method of laparoscopic surgery.

### BACKGROUND

Laparoscopic surgery which is also known as keyhole surgery or minimally invasive surgery (MIS) is a surgical technique in which operations in the abdomen are performed through small incisions provided in the abdomen wall. The incisions are typically of the order of 0.5-1.0 cm and provide the surgeon with access to the interior cavity for performing the necessary surgical operation. The abdominal cavity is typically inflated, using carbon dioxide, to increase the volume of the cavity so as to provide the necessary working and viewing space for the surgeon.

The surgical instruments are inserted through a provided cannula or trocar which creates an access point in the abdominal wall. Each operation typically requires a minimum number of such incisions and corresponding trocar placements to enable the use of a sufficient number of instruments. The control of the instruments is effected outside the body cavity. By using such minimally invasive surgical techniques as opposed to traditional open surgical procedures there are a number of advantages including the fact that the smaller incisions used amongst other factors reduces the pain of the operation and shortens recovery times for the patients. There is a further benefit in that there is often reduced risk of blood loss when compared to the traditional open surgery techniques which can reduce the necessity of blood transfusions and has obvious benefits for the patient.

Laparoscopic surgery by its very nature is based on minimally invasive principles and so it is accessed through the smallest necessary number of small diameter access points to the interior cavity. In keeping with the principles and intent of Laparoscopic surgery it is preferable for the surgeon to be able to carry out single operator procedures, implying that you would have only one operator of the instrumentation within the cavity. However, due to the number of instruments that may be needed for particular procedures and sometimes because of the limitations of current devices, there is often a requirement for two or more persons to access the interior cavity concurrently.

Furthermore often the actual site of the surgery is occluded by another organ that needs to be moved out of the field of view to allow access to the organ requiring treatment. In the context of traditional open surgery where access to the operating site is more accessible, this can be easily achieved and is often considered a conventional step in the operation. However with laparoscopic surgery, while the moving of occluding organs is still necessary, it is more difficult to achieve and has typically been achieved in one of two ways, both of which utilize rudimentary retraction devices.

Known retraction devices work on the principle of holding up the target from the outside in. They are a mix of single patient use (SPU) and reusable devices depending on the manufacture. Typically constructed of a metal shaft it is inserted via a 10/12 mm or 5 mm port and has various applicator end section designs depending on the manufacturer and model. These can for example be dimensioned to resemble finger blade type designs, or a simple triangular wedge shape. Some of these designs and end applicators are of metal construct but there are a number of inflatable types also available. These end applicator sections are designed and constructed in many different ways but essentially they all do the same thing that is to carry out a functional lift on the target organ. The target organ will depend on the actual operation being conducted. In the example of upper GI surgery and specifically the gastroesophageal junction and surrounding structures, the lift will usually be applied to the left lobe of liver to move it out of the field of view of the underlying target structures. These types of devices currently offered to the surgeon are advanced under the target organ, for example the liver, and the liver is then leveraged up and out of the field of view using the rigid lever. The device is then held in position by an assistant or some devices are fixed to an external support frame which acts as an aid to fix and hold it in position. In all current arrangements the retraction device is secured from the outside and most designs require a dedicated port/trocar throughout usage.

The use of dedicated ports suffers in that an additional incision is required, and as will be appreciated from above there is a desire in laparoscopic surgery to keep the number of incisions to a minimum. There is also a cost disadvantage of having to employ an additional port. Furthermore the maintaining of the retraction device in situ using a person requires that person to maintain a static hold for the entire procedure or certainly over prolonged periods of time causing fatigue to that operator. Fatigue usually leads to movement. This lack of operator control is not preferable as this can slow down or complicate the surgical procedure from the outset as they are relying on an assistant. Other device types require the assembly of an external fixing scaffolding around the operating table so as they can be fixed to it for the duration of the procedure. This can occupy valuable space and hinder the surgeon in his performance of the surgery. These devices constructs are typically reusable and require sterilization and maintenance.

Therefore there are a number of problems associated with existing liver retraction devices and their methods of use. There is also a distinct shortage of solutions to deal with emerging and challenging retraction in operations such as, but not restricted to, laparoscopic colon procedures. During this procedure the small bowel typically has to be maintained/retracted in a position out of the field of view of the target large bowel or colon.

### SUMMARY

These and other problems are addressed in accordance with the present teaching by provision of a retraction device for laparoscopic surgery that provides a lift from within the abdominal cavity and uses other features of the anatomy for supporting that lift during laparoscopic abdominal surgery. Such an arrangement has applications to deal with many of the situations where you need to move internal non target organs out of the field of view to access the necessary target anatomy to carry out successful resections and procedures.

In a first arrangement a retraction device is provided having an insertion configuration and an operational configuration. In the insertion configuration the device is collapsible such that the dimensions of the device can be reduced so as to allow the complete insertion of the device through a trocar or cannula into an internal abdominal cavity wherein it may be expanded to adopt the operational configuration. In the operational configuration the device is expanded and deployed to provide for the movement of desired organs so as to allow surgical access to other organs that would otherwise be occluded by the moved organs.

In a first arrangement the device comprises a flexible member that may be furled or rolled to adopt the insertion configuration. In the operational configuration the expansion of the device is effected by unfurling the flexible member to an extended configuration for contact with the desired organ so as to achieve the necessary movement, typically a lift, of that organ. Once the device is inserted into the body cavity it is controlled internally by the operator employing existing devices to move it into position and set it up to provide the retraction. This is as opposed to conventional retraction devices which require external manipulation to position the device and create the necessary lift or movement.

The flexible member may be coupled to an anchor element that provides for an internal anchoring of the device at a predetermined position within the abdominal cavity. In a first arrangement during the insertion configuration the flexible member may be furled about the anchor element so as to reduce the diameter of the device and allow its insertion through one of the available 0.5 cm to 1.0 cm Trocar access insertion points. Once received within the abdominal cavity the flexible member is unfurled or expanded to increase the surface area of the device to its operational configuration.

The flexible member desirably comprises a contact surface which operationally is in contact with the desired organ. The retraction device is desirably at least partially flexible so as to allow a lift of that organ out of the working space required by the surgeon.

The contact surface may be textured or otherwise treated to increase the coefficient of friction of that surface so as to increase the grip between the device and the desired organ. In a first arrangement a plurality of mechanical anti-slip elements are provided on the contact surface. In another arrangement, the contact surface is provided with an adhesive surface to provide a temporary adhesive bond between the contact surface and the desired organ.

On effecting lift of the desired organ the retraction device may be secured in place in a number of different ways. The use of a flexible coupling to connect the contacting surface to a support is particularly advantageous. In a first arrangement, the device is coupled to a suture which may be passed from the internal peritoneal cavity externally through the abdominal wall and secured externally via a tether placed on an outer surface of the abdominal wall or other portion of the torso. The suture is desirably secured to the tether as the lifted organ is acting downwardly under the effect of gravity. It will be appreciated that in the context of laparoscopic surgery that the abdomen is inflated and therefore the abdominal wall provides a substantially rigid structure for securing the tether against so as to counter any movement and retain the retraction device in situ.

In another arrangement the retraction device is secured against an inner surface of the abdominal wall using a securing member such as a surgical screw or the like. While the surgical screw will be considered a rigid element, at least a portion of the retraction device is desirably flexible to allow a flexing of the retraction device to adopt its secured position.

In another arrangement such securing is effected using an adhesive coating to bond at least a portion of the retraction device to an inner surface of the abdominal wall. The adhesive surface could also be provided in intimate contact with the target organ as well as or alternatively to the inner peritoneal abdominal wall so as to improve the securing of the device to the target organ. The adhesive will be considered at least partially flexible.

The anchor, if provided, is desirably dimensioned to be receivable within a predetermined anatomical feature, where once inserted, the weight of one or more organs acting on the anchor will retain the anchor in situ. For example in the assisted lift of the left lobe of the liver, the anchor may be designed and dimensioned to be receivable within a fissure provided between the left lobe and caudate lobes of the liver. Once positioned within the fissure, the weight of the left lobe acting downwardly on this proximal section of the retraction device will maintain the anchor in location. The more distal flexible member that is coupled to the anchor may then be pivoted relative to the anchor to achieve the necessary lift of the distal portions of the left lobe so as to provide a surgeon with access to the gastroesophageal junction and surrounding structures in this example.

In another configuration the flexible member may be dimensioned to envelop a portion of an organ at two sides thereof so as to effect a lift of that organ out of the field of view of a surgeon. In such an arrangement the device is secured at two locations in the form of a hammock, the organ being disposed between each of the two locations. An example of the use of such a configuration would be in the lift and or retraction of small bowel during a Laparoscopic Colon/Bowel procedure.

These and other features of the present invention will now be described with reference to an exemplary arrangement thereof which is provided to assist in an understanding of the teaching of the invention but is not intended to be construed as limiting the invention to the exemplary arrangements which follow.

### BRIEF DESCRIPTION OF THE DRAWINGS

The present invention will now be described with reference to the accompanying drawings in which:

FIG. 1A is an example of a retraction device in an insertion configuration in accordance with the present teaching.

FIG. 1B is an example of the retraction device of FIG. 1A in an operational configuration.

FIG. 2 shows deployment of a retraction device such as that shown in FIG. 1.

FIG. 3 is a detail view showing the retraction device located below the left lobe of the liver during use.

FIG. 4 is a sectional schematic showing the securing of the retraction device of FIG. 1 externally of the abdominal cavity.

FIG. 5 is an example of a flexible tether that may be used with the retraction device.

FIG. 6 is a perspective view showing the distal portion of the flexible member of the retraction device providing a lift of the left lobe of the liver.

FIG. 7 is an example of deployment of a retraction device which is secured internally off the abdominal cavity employing a chemical adhesive bond.

FIG. 8 shows an alternative means for securing a retraction device internally of the body cavity.

FIG. 9 shows an alternative configuration for an anchor.

FIG. 10 shows a further alternative configuration for an anchor.

FIG. 11 shows another arrangement for a retraction device in accordance with the teaching of the present invention.

FIG. 12 shows a flexible member portion of a device in accordance with the present teaching incorporating a plurality of straps that may be moved relative to a main body of the flexible member so as to at least partially encapsulate an organ.

FIG. 13 shows another arrangement of a flexible member provided in a 3-D configuration to assist in retention of a contacted organ.

FIG. 14 shows an example of a flexible member incorporating superelastic shape memory material elements provided for assisted deployment of the device.

FIG. 15 shows an arrangement of a flexible member incorporating a cut-out portion in accordance with the present teaching.

FIG. 16 shows the use of a drawstring about a perimeter of the flexible member in accordance with the present teaching.

FIG. 17 shows a further arrangement of a retraction device in accordance with the present teaching.

FIG. 18 shows a further arrangement of a retraction device in accordance with the present teaching.

FIG. 19 shows a collapsible anchor fabricated from a shape memory material in accordance with the present teaching.

FIG. 20 shows a deployment arrangement for effecting extension of a flexible member in accordance with the present teaching.

FIG. 21 shows another arrangement of a retraction device provided with first and second anchors.

FIG. 22 shows a first arrangement of an inflatable retraction device in accordance with the present teaching.

FIG. 23 shows a second arrangement of an inflatable retraction device in accordance with the present teaching.

### DETAILED DESCRIPTION OF THE DRAWINGS

Exemplary arrangements of retraction devices for laparoscopic surgery will now be described to assist in an understanding of the present teaching.

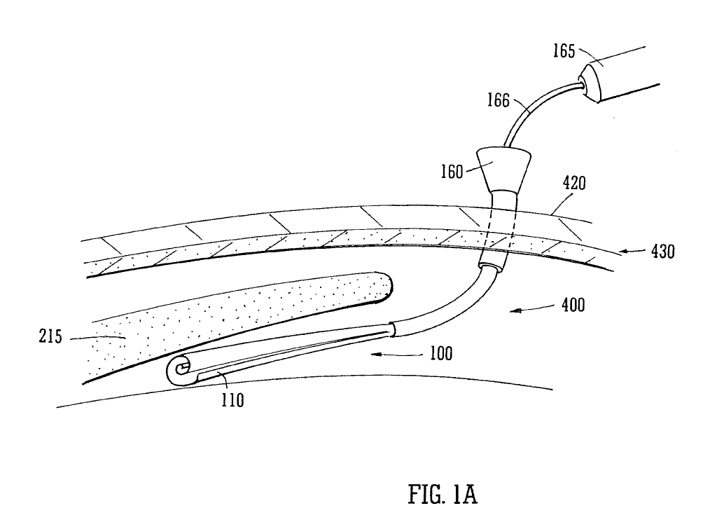

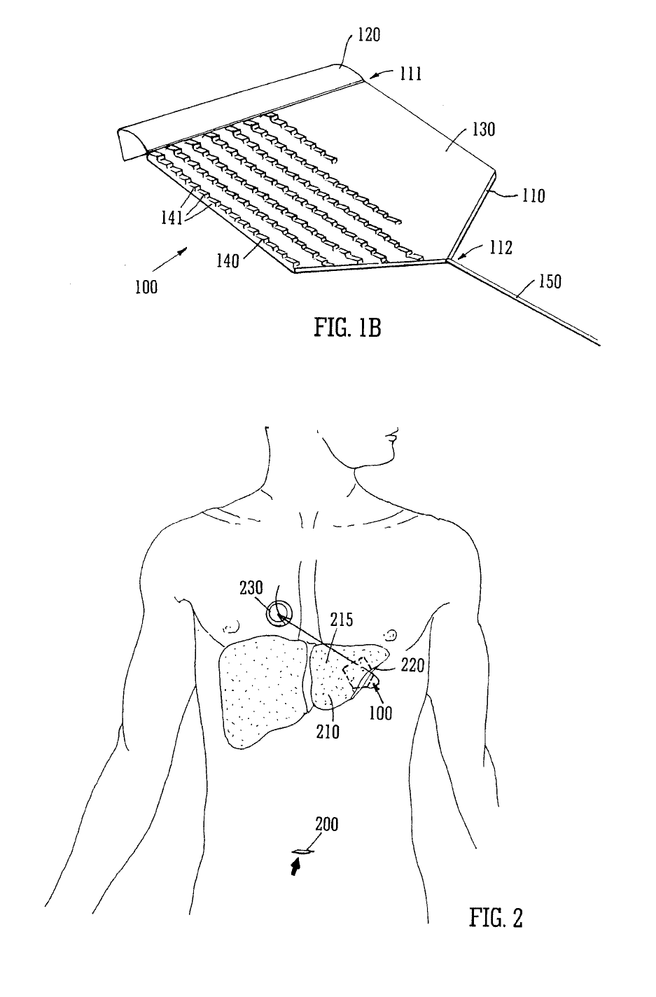

As shown in FIG. 1 a retraction device 100 in accordance with the present teaching is moveable between a first and second mode of operation. In a first arrangement shown in FIG. 1A, the device 100 is provided in an insertion configuration. In such a configuration the device 100 is collapsible such that the dimensions of the device can be reduced so as to allow the complete insertion of the device through a trocar 160 or cannula into the internal abdominal cavity 400 wherein it may be expanded to adopt the operational configuration such as shown in FIG. 1B. In this latter mode the device is expanded such that its surface area is increased so as to allow for the assisted lift and movement of desired targeted organs so as to allow surgical access to other organs behind them.

To provide for the adoption of each of the two modes of operation, the device 100 comprises a flexible member 110 that may be furled, rolled or otherwise compressed in size to adopt the insertion configuration. The flexible member may also be stacked upon itself in a vertical or horizontal stacking arrangement similar to a concertina whereby which each fold opens in the opposite direction to its neighbor. By stacking in a vertical stack, in the insertion configuration, the folds are typically arranged transverse to the ultimate longitudinal plane of the operational flexible member. In a horizontal concertina stacking arrangement, the folds will be arranged to be parallel with the ultimate longitudinal plane. In the operational configuration the expansion of the device is effected by expanding the flexible member 110 to an extended configuration for contact with the desired organ so as to achieve the necessary lift of that organ. In the arrangement of FIG. 1, the flexible member 110 is coupled to an anchor element 120 that provides for an internal anchoring of the device at a predetermined position within the abdominal cavity.

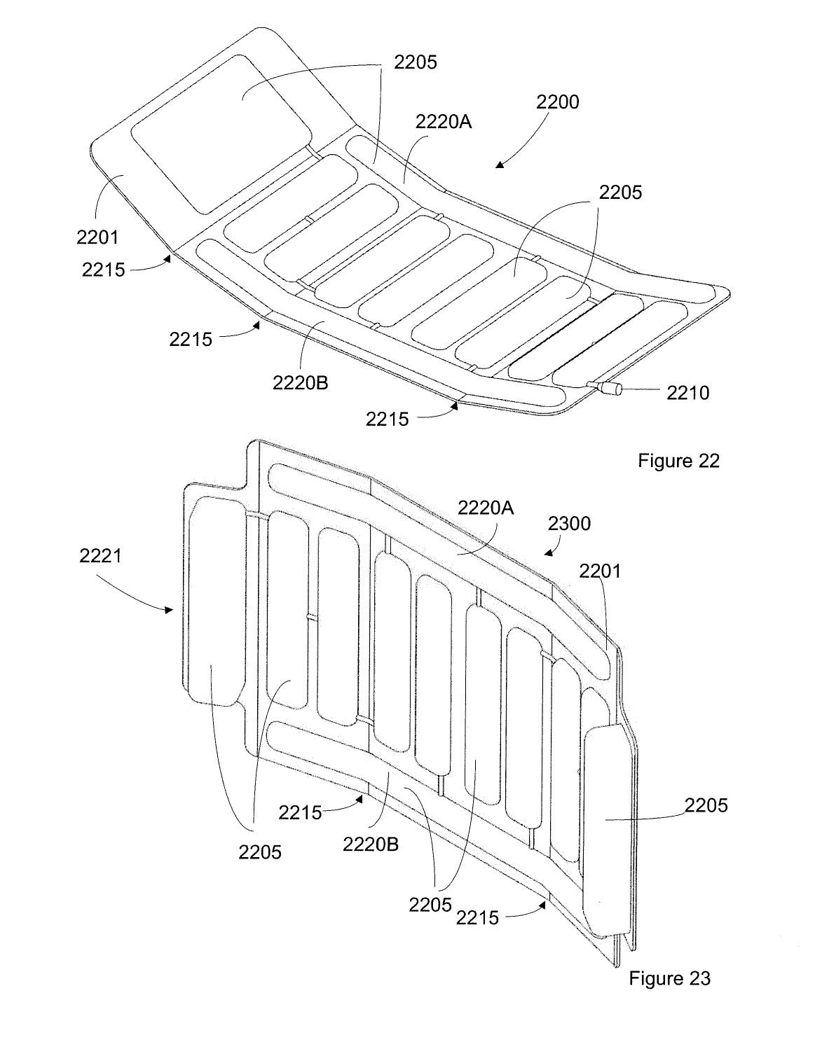

In the arrangements of FIG. 1 during the insertion configuration, the flexible member 110 may be furled about the anchor element 120 so as to reduce the diameter of the device and allow its insertion through the available 0.5 cm to 1.0 cm Trocar access insertion points. The insertion of the device 100 through the available trocar 160 is desirably achieved using an insertion tool 165 that includes a shaft 166, optionally a flexible shaft that can be used to bias the device 100 through the trocar and into the cavity 400. Once received within the abdominal cavity, the distal flexible member is unfurled or expanded to increase the surface area of the device to its operational configuration. This expansion could be provided by a balloon or other inflation mechanism controlled by the insertion tool 165. If such an arrangement is provided, the device 100 may include one or more balloon ribs which are configured such that on inflation, they will expand to provide the necessary structural change of the device into its operational configuration. These ribs, if provided, will typically run longitudinally along the flexible member 110. The ribs could be inflated using an air supply or by filling the ribs with a liquid. FIGS. 22 and 23 show two examples of such an inflatable retraction device 2200, 2300 whereby the anchor is integrated with the flexible member to provide a single component device. In these exemplary arrangements, an inflatable sheet of material 2201 is provided, which has a plurality of chambers 2205 which may be inflated using, for example, CO₂ or saline. This can be introduced into the device subsequent to insertion of the device into the abdominal cavity through coupling of a separate tool to a valve 2210 or the like. The sheet 2201 also comprises multiple hinge points 2215 which allow the flexing of the device to adopt a curved shape. In use, the device 2200, 2300 is positioned in roughly the desired location within the abdominal cavity, and inflation begins. In the exemplary arrangements illustrated, the chambers are provided in two sets of different orientations. A first set 2220, comprising top 2220A, and bottom 2220B chambers extending longitudinally in a direction substantially parallel with a longitudinal axis of the device, provide rigidity to the flexible member and act as a balance against the other inflation chambers and force the sheet into a more open configuration, giving a better seal against the abdominal wall. The second set 2221 extend in a direction substantially transverse to the longitudinal axis and again serve to stiffen the flexible member at defined locations to improve its capacity to abut against intended organs and control their movement. One or more of the individual chambers could have larger volumes than others of the chambers. In this way when inflated these larger volume chambers may project higher above the surface of the flexible member so as to provide a preferred contact surface for an organ or tissue element. This may be advantageously employed in anchoring the device at a desired location such that the anchor element is formed from an individual chamber. Typically, the anchor element if so provided is a chamber proximal to a perimeter region of the device.

An elastomeric material could be used around the perimeter of the device which would give the advantage of the device being able to conform to variations in anatomy, thereby improving the seal. While this has evident benefit in laparoscopic colonectomy procedures where it would prevent the small bowel from spilling into the working field, the use of such a device need not be limited to but could be applied to any procedure where the small bowel or other organs need to be retracted from the operating field.

Referring back to FIG. 1, the flexible member 110 may comprise of a contact surface 130 which operationally is in contact with the desired organ. As shown in FIG. 1B, the contact surface 130 may be textured or otherwise treated to increase the coefficient of friction of that surface so as to increase the grip between the device and the desired organ. In the example of FIG. 1B, a plurality of mechanical anti-slip elements 140 are provided on the contact surface 130. In this example they are shown as a plurality of ridges 141 that are arranged a plurality of parallel rows extending along the longitudinal axis of the flexible member 110. These mechanical slip surfaces are one example of means for maintaining contact between the flexible member 110 and the target organ.

In another arrangement, the contact surface is provided with an adhesive on the surface to provide a temporary adhesive bond between the contact surface and the desired organ. The provision of such a sticky surface provides for a high coefficient of friction without permanently adhering the flexible member to the surface of the structure/organ to be lifted. Exemplary adhesives that could be useful within this context include those known as buccal adhesives which provide a temporary bond but can be removed without damage to the contacted organ. In the arrangements heretofore the flexible sheet 110 has been described as being adhered to an anchor. The anchor provides for a securing of the flexible sheet against an anatomical feature so as to allow for the lift of the occluding organ. By providing the flexible sheet with an adhesive contact surface, the use of such an anchor may be obviated in that an intimate contact between the flexible sheet and the occluding organ is maintained through the use of the adhesive throughout available surface of the occluding organ. On completion of the necessary surgical procedure, the flexible sheet can be peeled away from the organ, collapsed to its non-operational configuration and removed from the body cavity. The removal of the device from its contact with the organ can be achieved through use of a temporary adhesive whose adhesion qualities may be formulated to diminish with time, or whose adhesion level is sufficient to provide for movement and securing of the organ but which on application of an external peel force will break contact with the organ. It may be designed to have any combination of the two formulas described to adhere and peel away.

The flexible member/substrate will typically be provided from a polyurethane or silicon sheet/web/net and/or may be provided with some self deployment capability in the form of for example a balloon integrated into its construct, as was described above with reference to the ribs that are inflated on deployment. It will be understood that to allow for the subsequent removal of the device once the operation is completed that it is necessary for the device to be collapsible again so as to allow its removal from the internal cavity 400. If the device is of an inflated variety it is necessary to include a valve to allow for release of that air/liquid from within the device so as to allow for its subsequent collapse and removal.

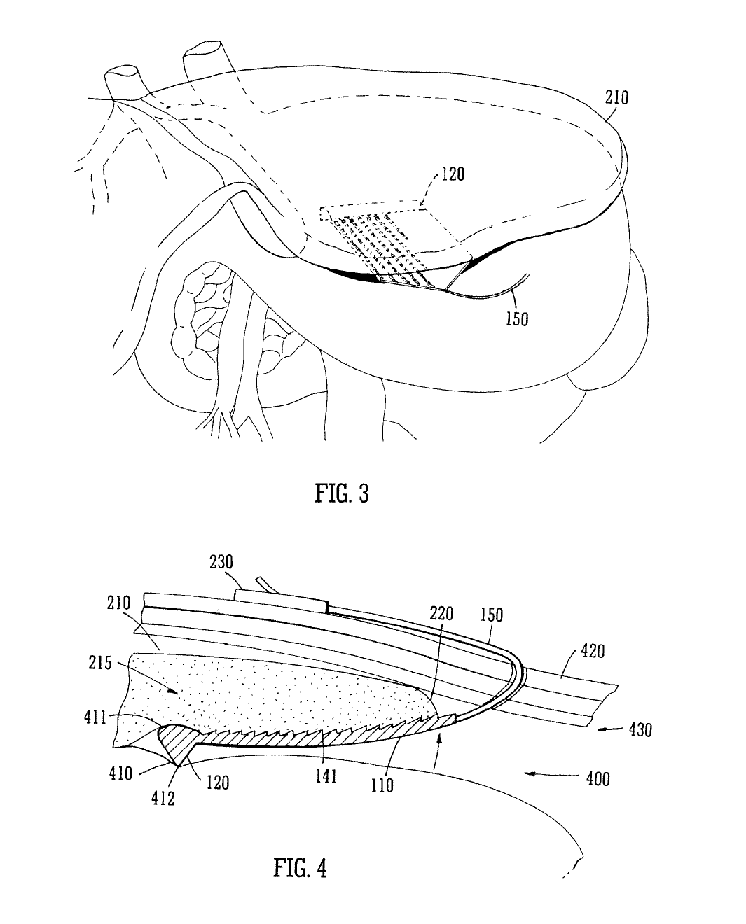

In the arrangement of FIG. 1, the flexible member has an anchor proximal portion 111 and an anchor distal portion 112. The anchor proximal portion is secured to the anchor. As the device of this arrangement is intended to be pivotable about the anchor 120, the distal anchor portion 112 is coupled to a suture 150 which will form the basis of the pull from an external lift to be fixed outside the patient, as will be evident from the following discussions of FIGS. 2 through 6. The suture 150 could be integrally formed with the device 100 or could be provided in situ by the surgeon through an eyelet or other coupling mechanism that is provided at the anchor distal portion 112. The suture may then be passed from the internal peritoneal cavity externally through the abdominal wall and secured externally via a tether.

As shown in FIG. 2 through 6, a retraction device 100 in accordance with the present teaching provides for the maintaining of the positioning of desired non-target organs out of the working environment required by a surgeon to allow the surgeon access to the target organs that would otherwise be occluded. The device is intended for use in minimally invasive surgery and is inserted into the abdominal cavity 400 (FIG. 4) through an incision 200 provided into an inflated abdominal cavity. In accordance with standard surgical techniques a trocar or cannula is used to ensure that the side walls of the abdominal cavity are not damaged by subsequent passing of surgical instruments into and from the abdominal cavity and to provide for ease of access.

In the examples figuratively shown, the device is used for movement of the left lobe 210 of the liver so as to provide access to surgical target organs below. The left lobe 210 has a proximal portion 215 near the caudate lobe and a distal portion 220 which normally covers the underlying anatomy, the gastroesophageal junction. Using a device such as that presently provided the left liver lobe distal portion 220 is lifted upwardly and away from the gastroesophageal junction and surrounding tissues.

To achieve this lift, the anchor 120 is dimensioned to be received within an anatomical fissure 410 (shown in FIG. 4) that is defined between the caudate lobe and the left lobe. On deployment of the device, the anchor 120 is located in that fissure and the weight of the left lobe proximal portion 215 acts downwardly on the anchor and retains the anchor in position. As shown in FIG. 4, the anchor 120 may be provided in any one of a number of different geometries depending on its desired positioning within the body. In this exemplary arrangement the anchor is wedge shaped having an arcuate upper surface 411 that is operably provided in contact with the liver 210 and which acts downwardly onto a ridge 412 that is located within the fissure 410. On location of the anchor within that fissure, the flexible member 110 of the retraction device is pivotable about the anchor so as to effect a lifting of the left lobe of the liver out of the working space required by the surgeon.

On effecting the lift of the desired organ, in this case the liver, the retraction device may be secured in place in a number of different ways. In a first arrangement shown in FIGS. 1 through 6, the device 100 is coupled to a suture 150 which may be passed from internally to externally through the abdominal wall and secured externally via a tether 230 placed on an outer surface 420 of the abdominal wall 430 or other portion of the torso. The suture is desirably secured to the tether and as the lifted organ is acting downwardly under the effect of gravity, as the abdominal wall is inflated and rigid, the tether will counter any movement and retain the retraction device in situ.

In this way the retraction device and the achieved retraction will then be secured and tied up outside the patient. The use of a straight needle and suture which will be passed through an eye or other coupling arrangement in the flexible member 110 provides for complete retraction and security of the occluding organ out of the surgical field of view of the surgical target anatomy without the need for or occupying any additional trocar. Furthermore as the device is self-consistent there is no requirement for additional personnel or external scaffolding to maintain the device in location or to maintain the lift as was required by prior art arrangements. Once inserted into the abdominal cavity the device is completely received within the cavity.

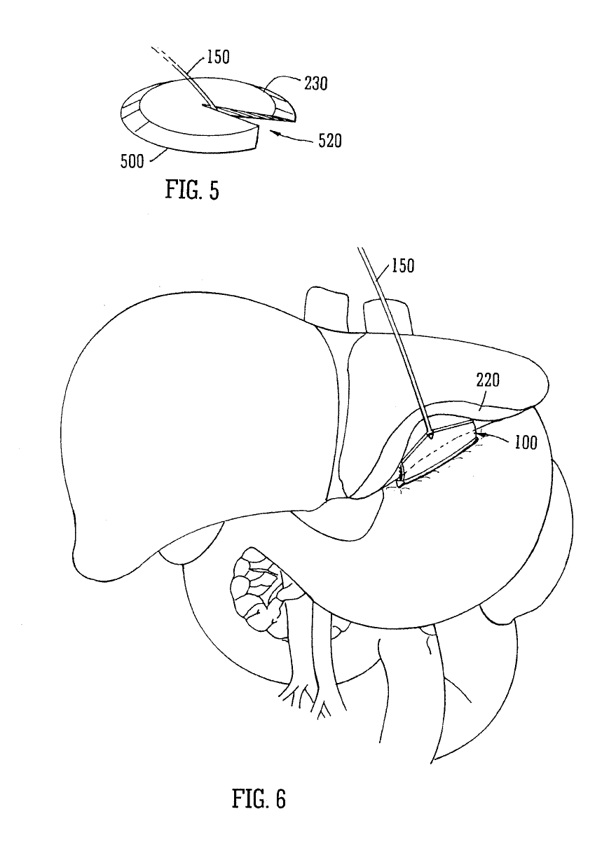

As shown in FIG. 5, the tether 230 desirably comprises a body contacting surface 500 which is sufficiently large in surface area to distribute the weight of the lifted organ across a wide area of the abdominal outer surface so as it doesn't bow in and compromise the lift effect. A cut away 520 in the tether provides for ease of location and securing of the suture 150 against the tether though of course other arrangements for securing the suture externally against the body of the patient could also be used.

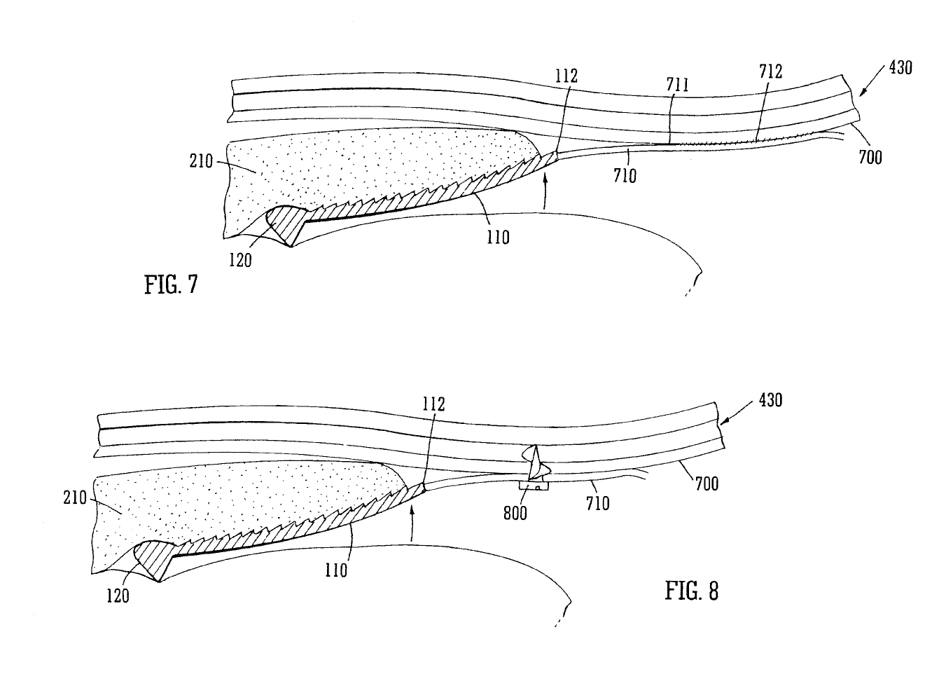

In others arrangement shown in FIGS. 7 and 8, where similar reference numerals are used for similar components to that described before, the retraction device is secured against an inner surface/peritoneal sheet 700 of the abdominal wall. In these arrangements the device comprises an abdominal wall contact portion 710 that is provided adjacent to the anchor distal portion 112 of the flexible member 110. This may be dimensioned to have the same width as the flexible member and as is desirably formed as an integral component of the device 100. As the abdominal wall contacting portion will also need to be inserted into the cavity, it is important that this too can be collapsed to allow it to pass through the trocar's internal bore provided in the abdominal wall.

In contrast to the arrangement of FIGS. 1 to 6, in these two examples once the desired lift of the organ is achieved, the device 100 is secured against an inner wall of the body as opposed to the outer wall. In the arrangement of FIG. 7 the abdominal wall contacting portion 710 includes a contact surface 711 provided with an adhesive 712 that allows for the temporary bonding of the surface to the abdominal wall to maintain the desired lift. In the arrangement of FIG. 8, the abdominal wall contacting portion 710 is contacted against and secured to the abdominal wall using a securing member 800 such as a surgical screw or the like.

The anchor described heretofore has been wedge shaped. It will be appreciated that such geometry is exemplary of the geometries that could be used within the context of the present teaching. The anchor could be fabricated from a substantially rigid material, but having a diameter small enough to allow its presentation through the trocar and into the cavity. In another configuration the anchor could be fabricated from a flexible material but having an internal cavity which when filled with air or liquid will adopt a rigid configuration. In such a balloon type construct, the anchor will be deflated on insertion and removal from the body cavity. On receipt within the cavity, inflation of the anchor will allow it to adopt its operational configuration for subsequent usage.

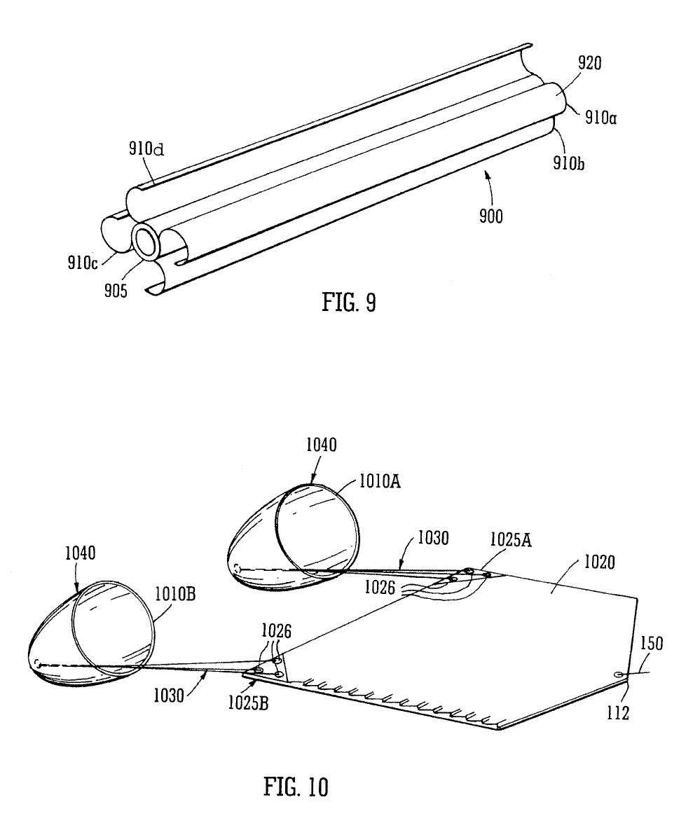

FIGS. 9 and 10 show other exemplary geometries that may be usefully employed within the construct of anchors for use with devices in accordance with the present teaching. As shown in FIG. 9, an anchor 900 comprises a central rib 905 that defines the length of the anchor. A flexible sheet, while not shown in this Figure, could be attached along the length of the rib 905—similarly to that described with reference to FIG. 1B. A plurality of legs 910, in this exemplary arrangement 4 legs 910a, 910b, 910c, 910d, are provided and extend radially from the central rib. Each of the legs 910 provide a curved surface or arcuate surface that in this configuration curves in a clockwise direction (this being appreciated as being an exemplary arrangement) so as to provide an increased contact surface and configuration 920 that may engage with a desired anatomical feature to secure the anchor thereto. The provision of a plurality of legs can be usefully employed to provide multiple contact surfaces between the anchor and the anatomical feature. Such an anchor could also be usefully configured as was described with reference to other anchor arrangements to include an inflation mechanism whereby the rigidity of the anchor is increased by inflation of internal cavities within the anchor.

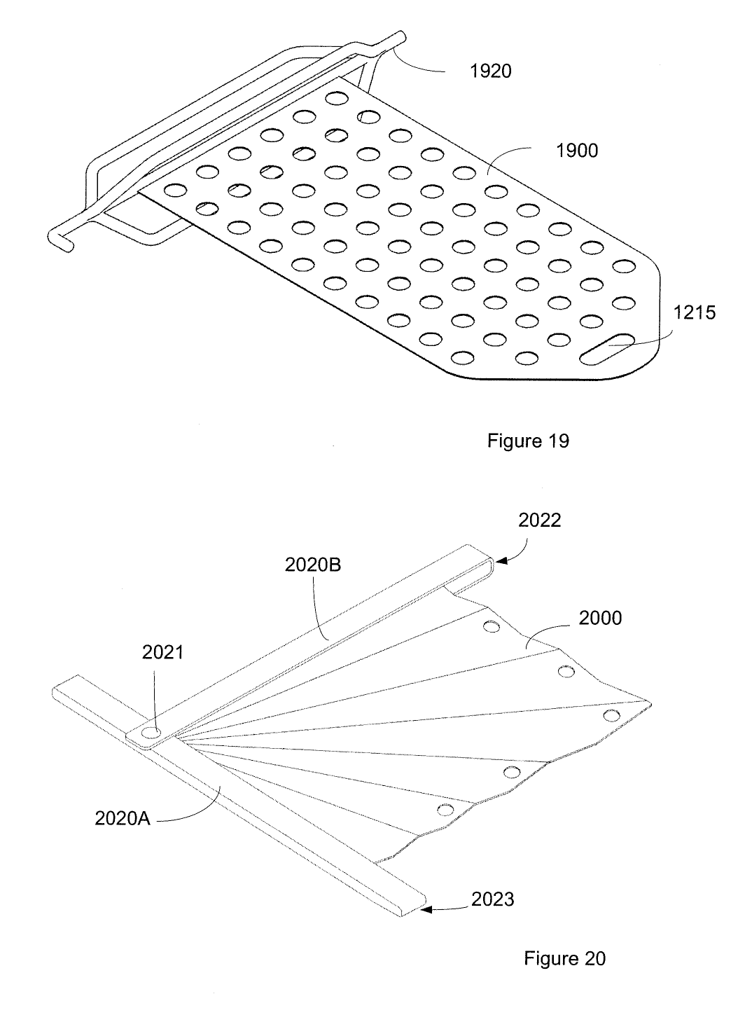

FIG. 10 shows a further arrangement that could be usefully employed within the context of the present teaching. Differing from the previous anchor arrangement, FIG. 10 shows where a plurality of anchors 1010A, 1010B are used to effect a securing of the flexible sheet relative to an anatomical feature within the body cavity. In FIG. 10, two anchors are shown but it will be appreciate that two or more could be equally used depending on the actual intended deployment area of the device. In FIG. 10 a flexible sheet 1020 is coupled at two edge portion 1025A, 1025B to the anchors through multiple contact points 1026. In this arrangement the anchors are coupled by a plurality of sutures 1030 or other tethering arrangements. By using a plurality of sutures and distributing them about the edge portions 1025 it is possible to distribute the tension across a wider surface area than simply using one contact point between each anchor and the flexible sheet. By coupling using a flexible member such as a suture it is possible to locate the anchors in more advantageous anatomical features than may be otherwise possible if they were in intimate contact with the flexible sheet. The arrangement of FIG. 10 shows the two anchors in a bell or cup geometry—with the anchors arranged relative to the flexible sheet such that a mouth 1040 of each cup is proximal to the sheet 1020. It will be understood that the dimensions of each anchor will be optimized for the intended deployment region. FIG. 19 shows a further modification to an anchor arrangement whereby a support web 1900 is attached to a self expanding nitinol anchor 1920, nitinol being an exemplary arrangement of a shape memory material that may be used within this context. The form of the support web 1900 is shown in FIG. 19 as being in the form of an open mesh, but it will be appreciated that other configurations such as for example a closed mesh or a sheet of polymer with raised profiles to provide grip on surface of the liver or other organ could be provided. The arrangement of FIG. 19 shows the self expanding anchor 1920 in its expanded state. During insertion, the anchor would be constrained within a thin walled tube to minimize its profile and allow for insertion through a trocar. The device would be inserted through the trocar into the body with the mesh 1900 wrapped around, within or alongside the anchor to reduce insertion profile. Once unfurled the anchor would be inserted to the desired location under the liver or other organ. The constraining tube would then be removed allowing the anchor to expand to its shape set profile, anchoring the support web in place. A suture could then be placed at the dedicated suture port 1215, or indeed any point of the support web if a mesh is used, which would allow for greater flexibility in the size range of livers or other organs the device could deal with. The suture could then be anchored by passing it through the abdominal wall or onto an internally placed anchor point.

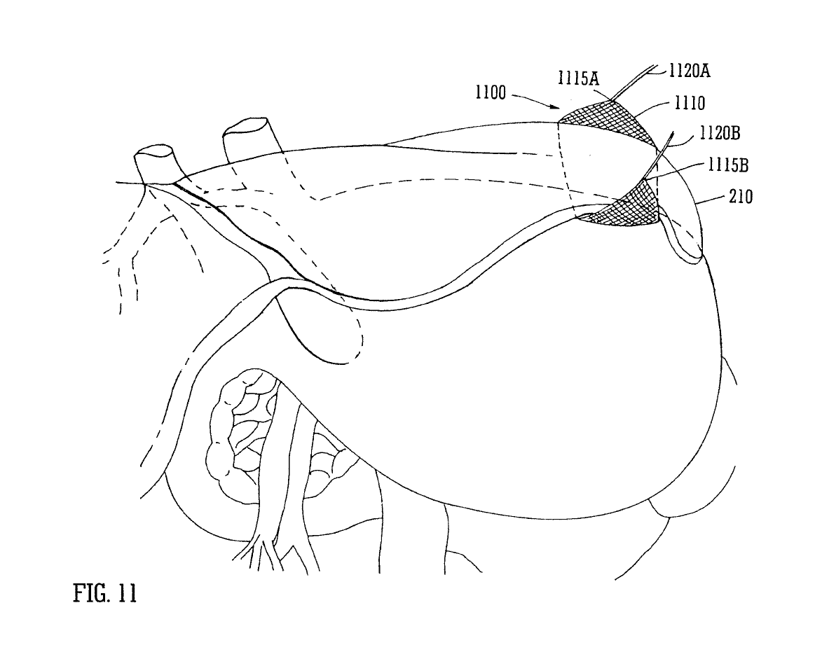

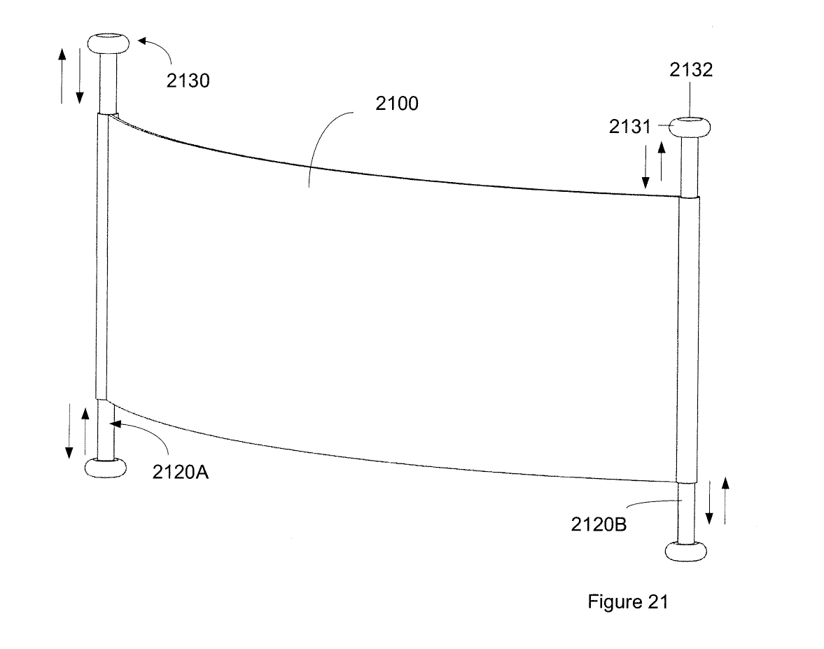

In the arrangements described heretofore the device has included an anchor dimensioned to be receivable within a predetermined anatomical feature, where once inserted, the weight of one or more organs acting on the anchor will retain the anchor in situ. The example given was in the assisted lift of the left lobe of the liver, where the anchor may be dimensioned to be receivable within a fissure provided between the left lobe and caudate lobes of the liver. Once positioned within the fissure, the weight of the left lobe acting downwardly will maintain the anchor in location. The flexible member that is coupled to the anchor may then be pivoted relative to the anchor to achieve the necessary lift of the distal portions of the left lobe. In other applications requiring retraction and/or with other applications extra to requiring a movement of the liver, it may not be feasible to anchor the device within anatomical features. In such arrangements it is desirable to provide the device having a flexible member dimensioned to envelop a portion of an organ at two sides thereof so as to effect a lift of that organ out of the field of view of a surgeon. FIG. 11 shows an example of such a hammock arrangement 1100 whereby a flexible sheet 1110 includes a first 1115A and a second 1115B contact location. The first and second contact locations are provided at opposing ends of the sheet 1110 such that the organ to be moved—in this case the exemplary left lobe of the liver 210, may be disposed between each of the two contact locations (another practical example may be the small bowel in a laparoscopic colon procedure). By providing the flexible sheet with sufficient width and length contact between the flexible sheet 1110 and the lifted organ 210 is maintained. The lifting of the sheet at each of the two contact locations 1115A and 1115B can be used to effect a movement of the organ away from the target organ(s) that is occluding the target surgical site. Each of the two contact locations are desirably coupled to a tether—such as a suture 1120. The two sutures 1120A, 1120B could be independently tied or otherwise secured in a fashion similar to the described before. In another arrangement the two could be secured relative to one another such that a tensioning of a first suture would effect a corresponding tensioning of the second to provide for distributed and even tension across both contact locations. It will be appreciated that the hammock arrangement of FIG. 11, provides for an anchoring of the device at opposing ends of the flexible member. FIG. 21 shows an alternative arrangement whereby first and second anchors 2120A, 2120B are provided at opposing ends of a flexible member 2100. Such an arrangement is particularly advantageous for use in small bowel procedures. In this configuration the flexible member 2100 of the retraction device may be formed from thin film, web, or mesh suspended between the two sprung loaded supports 2120A, 2120B which enable anchoring/fixation of each side of the retractor. While shown in FIG. 21 as each end of each support being moveable in a sprung-loaded manner, it will be appreciated that this is illustrative of the exemplary arrangement and none or more of these supports may be provided with a biasing element to allow their length to be modified during use. A soft material such as low durometer polyurethane or silicone may be molded over an end 2130 of each support to effect less traumatic fixation. These end portions 2130 are desirably provided with a bulbous configuration having curved side walls 2131 terminating with a planar upper surface 2132 which in use will abut against an anatomical feature or organ within the abdominal cavity. By providing the curved side walls 2131, there is less risk of tearing or embedding of supports within tissue. The sprung loaded supports may be designed to provide very slight or significant resistance depending on the procedural requirements. The sprung loaded supports may also be replaced by eyelets to enable suturing as a method of fixation. The flexible member 2100 that forms part of the retractor device may be wrapped around the supports 2120 to effect a low profile for delivery laparoscopically. Each of the overmolded supports 2120 can be offset axially in the low profile to enhance deliverability. The flexible member may be preformed to provide an optimal in vivo profile such as the curved profile that is evident in FIG. 21.

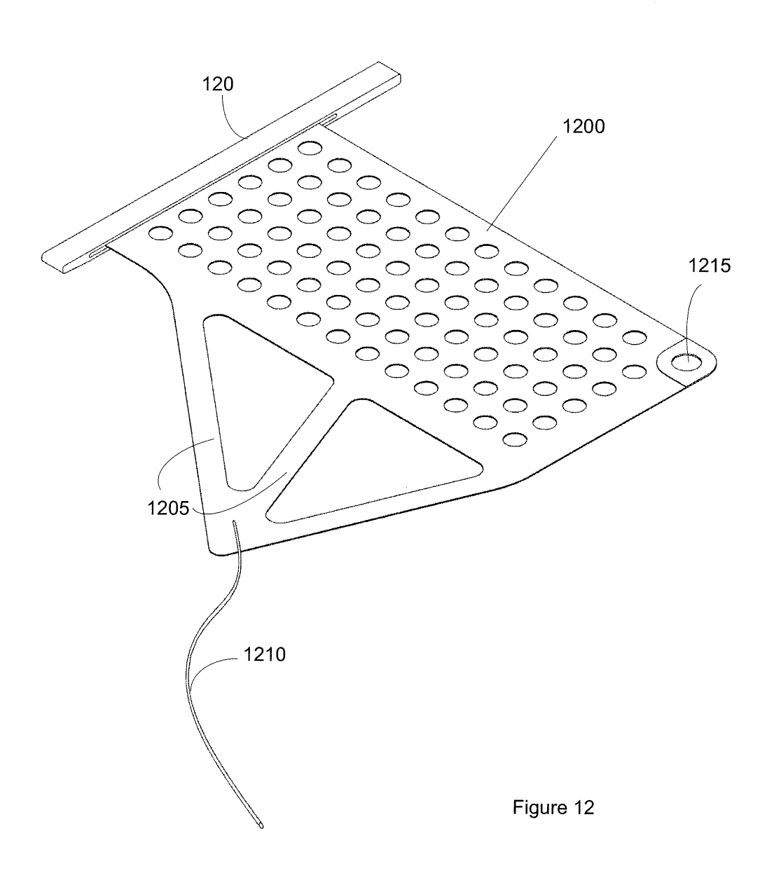

FIG. 12 shows a modification to the flexible member described heretofore. In this arrangement the flexible member is provided in the form of a web or mesh 1200 having one or more straps 1205 coupled thereto. The straps 1205 are linked to a suture strand 1210. Once the mesh is placed under the organ to be retracted, the suture 1210 is passed through an eyelet 1215—shown in FIG. 12 as being on the other side of the web to the location where the suture is coupled to the straps. By then pulling the suture, the straps are configured to encircle the organ, passing around and over the organ to secure it in place. The suture can optionally be sutured to the abdominal wall or passed out through the abdominal wall. The benefit of this approach is that it provides a mechanism for preventing the liver from slipping off the mesh or web 1200 in that the organ is retained through a tightening of the straps 1205 about the organ. The straps may be elastic to accommodate a variety of target organ geometries. The straps may be integrally formed with the material used for the body of the flexible member or may be formed separately and attached to the main body. Typical materials that may be employed for this specific flexible member, and other configurations or embodiments as described herein, include thermoplastics such as polyurethane, a polypropylene such as PET or a silicone elastomer. While it is not intended to limit the present teaching to any one set of parameters or values, it will be understood that in providing surgical devices that are being used to provide a surgeon with access to specific locations during surgery that it is important that the actual device is dimensioned so as to not occlude the actual view that it is trying to create. For these reasons it is desirable to form the flexible member as thin as possible and the use of the materials heretofore described may be advantageously employed to fabricate a flexible member whose wall thickness would typically range from 0.0025 mm to 1 mm and more preferably from 0.025 to 0.1 mm.

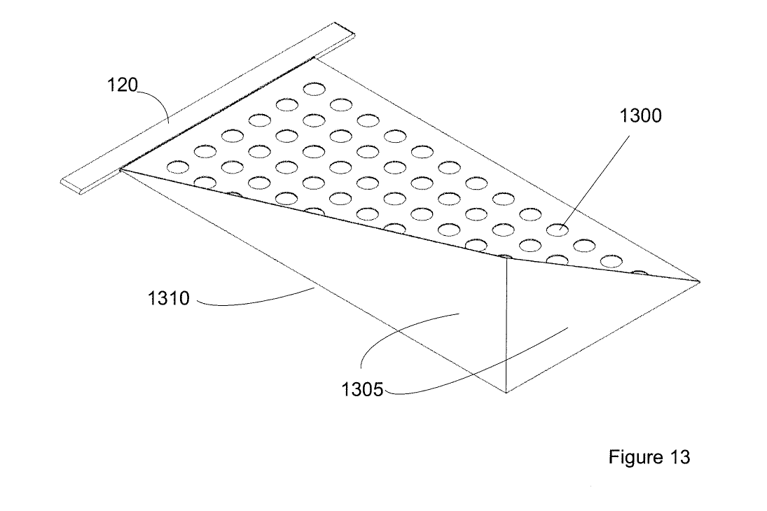

FIG. 13 shows a modification to such an arrangement whereby as opposed to defining a three dimensional shape web through a tightening of straps elements of the web, the web is preformed with a three dimensional shape in order to better encapsulate the organ being retracted. In the arrangement of FIG. 13, a mesh 1300 is provided having raised sides 1305 provided at an edge portion 1310 thereof, in the arrangement shown two raised sides are provided extending along the side and end of the mesh. The raised sides operably serve to provide a retention wall or pocket such that as the mesh is retracted the raised sides engage with and retain at least a portion of the liver or other organ, thereby preventing it from slipping off the mesh. This is particularly useful especially when the mesh is being retracted obliquely. A benefit of this approach over a flat or planar web is that the organ in encased more securely in the web. It will be appreciated that the shape shown here is for illustrative purposes but should not be seen as limiting the design. The shape could be stitched into the mesh from a separately formed element or the mesh may have a naturally embedded concave shape.

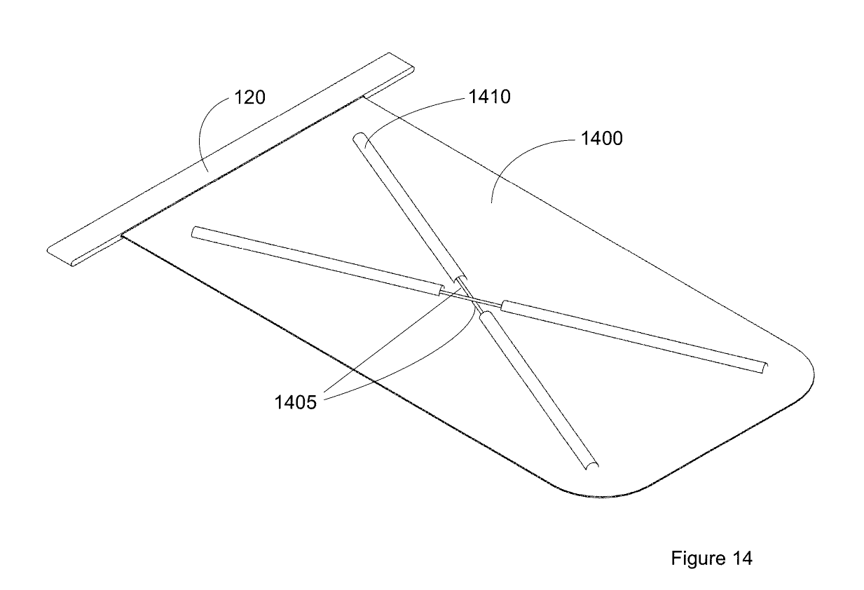

In another arrangement shown in FIG. 14 the flexible member is provided in the form of a web or mesh 1400 with additional nitinol supports 1405 embedded thereon or therein. In the exemplary arrangement a plurality of pockets or channels 1410 are provided on the mesh surface for receiving the supports 1405. It will be appreciated that the nitinol is an example of a shape memory material which as a result of its physical properties will tend to revert to a predefined shape on release of a force thereon. These supports 1405 ensure the mesh 1400 unfurls without the need for operator manipulation. In the exemplary arrangement illustrated, the supports are provided as two diagonal wires extending across the web 1400, but it will be appreciated that the location of the memory material or its configuration (for example sheet or wire) may be modified. One typical modification would be provision of a support about the perimeter or circumference of the flexible member to reduce the time needed to prep the device inside the body and provide an ease of use benefit. The elements could also be arranged to achieve a geometry such as that shown in FIG. 13 or to achieve a generally concave geometry also.

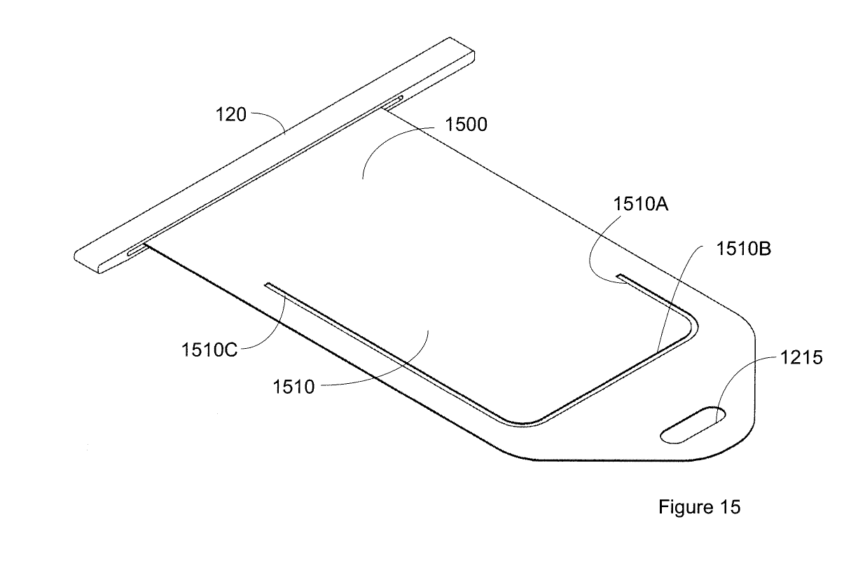

FIG. 15 shows a further modification of the flexible member, in this exemplary arrangement optimized for use in retraction of the left lobe of the liver. In this arrangement the flexible member is again provided in the form of a mesh 1500, but in this arrangement is provided with a cut-out section 1510. In the exemplary utility of retraction of a liver, on location of the mesh under the liver and a subsequent lifting of the mesh, some of the liver mass falls through the hole created by the cut-out 1510. The mesh once pulled back to where it is anchored is effectively encircling the tip of the liver. The benefit of this approach is that it provides a mechanism for preventing the liver from slipping off the mesh. The exemplary cut-out 1510 shown in FIG. 15 comprises first 1510A, second 1510B and third 1510C cut out lines which perforate the integrity of the mesh fabric and are arranged with the first 1510A and third lines 1510C being coupled via the second line 1510B with the second line 1510B forming a base of the cut-out and being arranged substantially perpendicular to the longitudinal axis of the mesh. By having the longer cut out 1510C provided on the right hand side of the mesh—in the direction extending outwardly from the anchor 120—the cut-out will provide a larger gap or aperture in this region for receiving the non-fixed edge of the left lobe. It will be observed from this exemplary arrangement that the cut-out is located in a region proximal to a tip of the mesh, in this arrangement adjacent to the eyelet 1215 that may be used to secure the mesh on effecting the retraction of the organ. The profile of the cut-out shown here is for illustrative purposes and it will be appreciated that it is not intended to limit the geometry of such a cut-out. This use of a cut-out could be combined with a drawstring which encircles the perimeter of the cut-out section and is used to provide lift to the mesh. The drawstring, if provided, would tighten the cut-out opening around the liver.

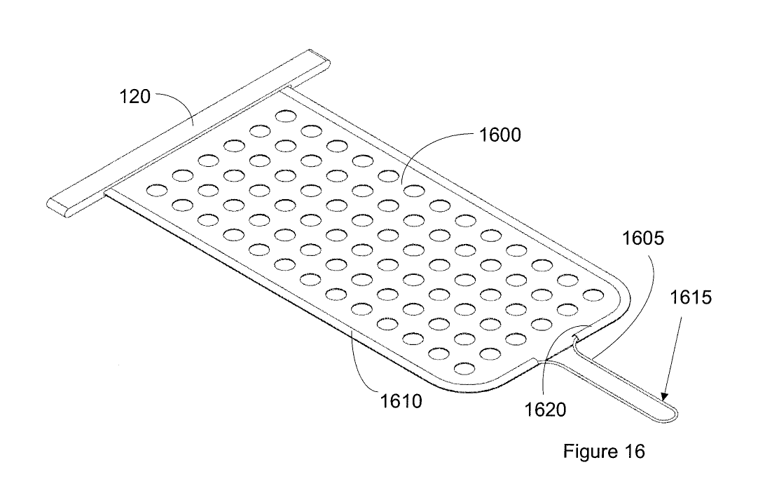

In another configuration of a flexible member, shown in FIG. 16, the flexible member is again provided in the form of a mesh 1600, but in this arrangement a drawstring 1605 is provided about a perimeter 1610 of the mesh 1600. The drawstring 1605 may be provided within a channel 1620, such that it is encapsulated and will not snag against organs or the like during use. The function of the drawstring is to tighten the mesh 1600 about the organ being supported. The drawstring may have a hook/clip disposed on its end 1615 such that the drawstring once taut can be attached directly to the internal cavity of the abdomen. Alternatively, the drawstring could be taken external to the abdominal cavity through for example a trocar or needle with suture catching capability. An advantage of this approach over a mesh without a drawstring is that the organ is more securely held by the mesh and does not tend to slip out of the mesh, especially if the mesh is pulled back at an oblique angle relative to the anchor element—not shown.

It will be appreciated that in the exemplary arrangements described herein that it is possible to use the rigid nature of the abdominal wall—arising from inflation of the abdominal cavity during laparoscopic surgery to hang or suspend the weight of a lifted or otherwise moved organ therefrom. A retraction device such as that provided within the present teaching may be inserted wholly or fully into the internal cavity through an available trocar and then provided underneath organs or other visceral anatomy to move them from their normal location where they are occluding other target areas that require surgery. Such insertion of the devices will be effected by a surgeon or other member of the surgical team. The devices, once inserted are fully contained within the cavity and their manipulation is effected within the internal cavity. This allows the surgeon to locate them relative to the desired target organs-secure them in position and then conduct the necessary surgery without requiring subsequent manipulation or control of the devices externally of the body. In this way there is no need for additional surgical team members to hold or retain the retraction devices externally of the body cavity—as was a requirement of prior art arrangements or for steep patient positioning using gravity to move non target organs out of the field of view.

A device provided in accordance with the present teaching will desirably comprise a contact sheet which may be expanded subsequent to insertion within the cavity. To allow for such expansion, it is desirable that the contact sheet is fabricated from a flexible material that would allow it to adopt a collapsed configuration during insertion into and removal from the body cavity. The flexible member is desirably formed of a material having a shape whose length is greater than its width. The material is arranged relative to the anchor so as to have a longitudinal axis substantially transverse to a longitudinal axis of the anchor 120. The width of the flexible material will typically substantially correspond with the length of the anchor element. The flexible material may be formed as a mesh having a plurality of apertures or features relatively large in dimension (for example approximately 1 to 30 mm, or more desirably from 2-20 mm) formed on the contact surface thereof. These holes or features operably allow the organ tissue to invaginate into the material allowing for improved grip between the mesh and the organ tissue. Examples of a flexible member incorporating such a plurality of apertures has been described with reference to FIGS. 16-19.

As was described above, the flexible member or sheet may be secured via one or more anchor points. Such anchoring if provided could be provided by exemplary arrangements such as:

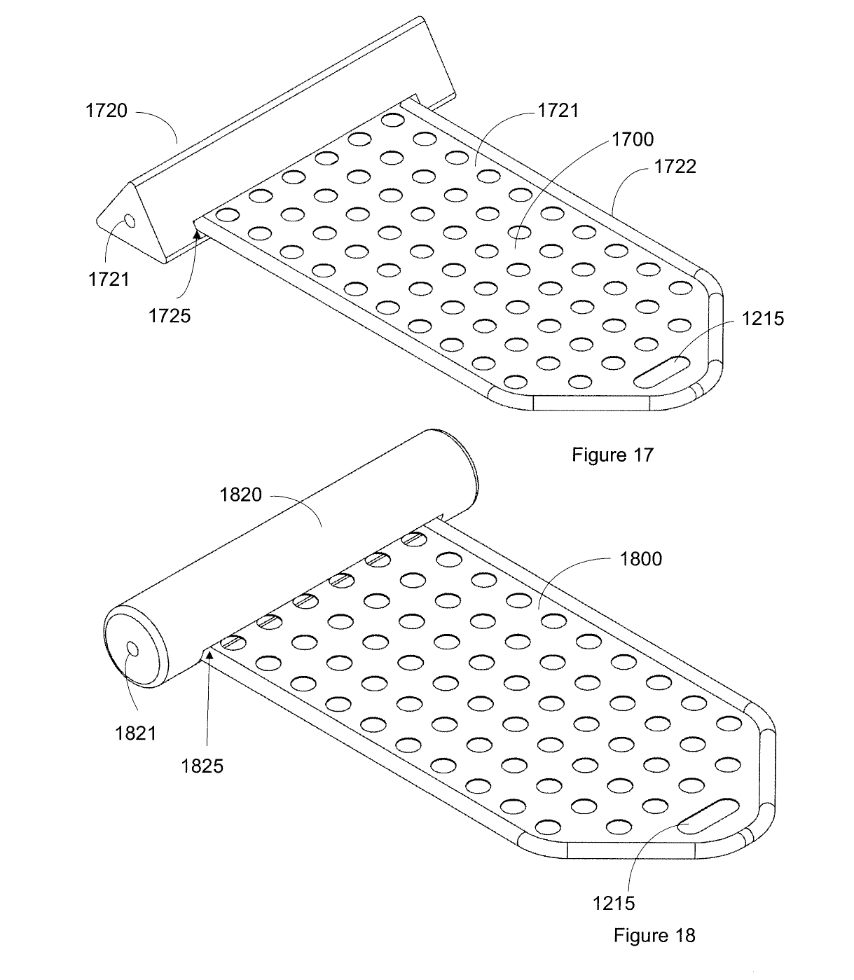

1. Cylindrical type embodiment that will provide a radial force and be of dimension and have materials properties that will allow it to be manipulated into place under the organ to be retracted. Suitable materials and configurations include balloon, expanding alloy/metal and self expanding foam, FIGS. 17 and 18 show examples of alternative exemplary anchoring arrangements, where in FIG. 17 an anchor 1720 having a substantially triangular profile is provided. A mouth region 1725 provided in one of the faces of the anchor 1720 defines an opening for receipt of at least a portion of the flexible member 1700 therein. By providing attachment of the flexible member 1700 within an interior volume of the anchor, the pivot point of the flexible member relative to the anchor can be more precisely defined. Furthermore the level of securing of the two to one another can be improved. In this exemplary arrangement of the anchor a contact point 1721 is provided which may assist in the movement or manipulation of the anchor 1720 to a desired location within the abdominal cavity. The flexible member 1700 in this exemplary arrangement comprises a body portion 1721 of a first thickness and a ribbed perimeter portion 1722 of a second thickness, the second thickness being larger than the first thickness. By providing this ribbed element 1722 about the perimeter of the flexible member 1700, on an unfurling of the flexible member the ribbed element biases the remaining portion of the flexible member to adopt and maintain the expanded configuration. While this may be provided as a solid element, formed during for example the manufacturing process, it is also possible to provide such an arrangement through provision of an inflatable chamber about the perimeter of the device with inflation being effected post insertion into the abdominal cavity. FIG. 18 shows a modification to the arrangement of FIG. 17 where a cylindrical anchor element 1820 is provided which similarly to the device of FIG. 17 provides a mouth 1825 within which a portion of the flexible member 1800 is received. In this arrangement however, the anchor is configured to operably receive substantially all of the flexible member through a retraction of the flexible member through the mouth region 1825 into an interior volume of the anchor. This retraction and subsequent extraction could be effected through use of a spring loaded mechanism or other arrangements which allow for the receipt of the flexible member into the anchor and subsequent deployment of the device by expansion of the member from the anchor volume. In an exemplary arrangement the deployment could be controlled such that a defined length of the flexible member could be extracted as needed. This controlled release could be achieved through actuation of a release member 1821 provided on an outer surface of the anchor 1820.

2. Separate anchoring shaped devices that are in turn attached to the flexible member.

In arrangements where the flexible member is provided with an adhesive surface, or where for example it is configured in a hammock type configuration, the use of dedicated anchors may not be required.

FIG. 20 shows a further modification to a retraction device in accordance with the present teaching. In this arrangement, the anchor 2020 is provided in a two-part construction having a first 2020A and a second part 2020B. The second part 2020B is coupled to and pivotable relative to the first part 2020A via a pivot point 2021. The first and second parts are each coupled to the flexible member such that movement of the two parts effects a corresponding movement of the flexible member 2000. During insertion of the device into the abdominal cavity, the first and second parts are axially aligned with one another by bringing an end 2022 of the second part towards a corresponding end 2023 of the first part. By moving these two ends towards one another the profile of the device is reduced and the flexible member 2000 is folded onto itself. The bringing together may be such as to have one of the two parts received into the other of the two parts. Once the device is provided through the trocar and into the abdominal cavity the first and second parts can be pivoted relative to one another. As each of first and second parts are coupled to the flexible member 2000, their movement apart effects an opening of the flexible member in a fan-like arrangement. The provision of first and second parts provides two anchoring positions in two different planes. When used for retraction of a liver, such a configuration allows the user to easily push the flexible member under the liver with advantages being increased support and ease of use in relation to deploying the flexible member under the liver. It will be appreciated that a device as provided in accordance with the present teaching may have a number of requirements.

It should be capable of being inserted into a body cavity through available laparoscopic entry ports.

On insertion it should be capable of being located relative to and secured against an organ to be moved.

It should be capable of providing assisted lift or movement of that organ.

It should be capable of being secured in place once that movement is achieved.

Addressing these in turn, an exemplary arrangement of a device delivery system has been described with reference to FIG. 1A. In that arrangement a push rod mechanism was described that provides for the insertion of the device into the body cavity through a pushing of a rod. In another configuration a balloon deployment catheter type system could be employed which could use catheter type technology to expand a two stage balloon on insertion.

The anchoring of the device could be achieved through use of one or more mechanical anchors and/or using a chemical bond to secure the device. Mechanical anchors could be employed using balloon techniques to provide rigidity on insertion within the body cavity. The geometry of the anchor will depend on the deployment location. While exemplary arrangements of coupling the flexible member to the anchor element have been described it will be appreciated that modifications to that heretofore described could include an arrangement whereby the flexible member extends obliquely and/or tapers outwardly from or inwardly to the anchor. Such an arrangement could be used in situations requiring shorter anchors relative to the width of the flexible member and/or could be used to bias the flexible member favorably relative to the anchor.

To provide the necessary lift it is desirable that the device has a contact surface that can be provided in contact with the organ to be lifted. Exemplary embodiments of a flexible sheet have been described with reference to high friction surfaces, adhesive coatings and the like. This sheet will desirably be provided in a collapsed configuration and on receipt into the body cavity will be expanded to adopt the operational configuration. Such expansion could be effected using balloon technology or by a simple unfurling or other type of expansion of a collapsed sheet or web of material.

Once movement is achieved it is necessary to hold that organ in situ until the operation is complete. Two general exemplary types of means for securing the device in situ have been described. In a first arrangement a suture and needle are used to pull the flexible sheet taught. The suture was either passed out through the abdominal wall and held in place through use of a washer type arrangement on the outer surface of the body wall which displaced the weight across a larger area or was held in place internally using for example self retaining mechanical screw fixed to the inner wall without passing right through the wall of the abdomen. In another technique a chemical bond was used to adhere the device to an internal surface so as to hold the device in situ.

While preferred arrangements have been described in an effort to assist in an understanding of the teaching of the present invention it will be appreciated that it is not intended to limit the present teaching to that described and modifications can be made without departing from the scope of the invention.

It will be appreciated that the exemplary arrangements or examples of devices have been described with reference to the Figures attached hereto. Where a feature or element is described with reference to one Figure, it will be understood that the feature or element could be used with or interchanged for features or elements described with reference to another Figure or example. The person of skill in the art, when reviewing the present teaching, will understand that it is not intended to limit the present teaching to the specifics of the illustrated exemplary arrangements as modifications can be made without departing from the scope of the present teaching.

The words comprises/comprising when used in this specification are to specify the presence of stated features, integers, steps or components but does not preclude the presence or addition of one or more other features, integers, steps, components or groups thereof.

1. A laparoscopic surgical retraction device having an insertion configuration and an operational configuration wherein in the insertion configuration the device is collapsible such that the dimensions of the device can be reduced so as to allow the complete insertion of the device through a trocar or cannula into an internal abdominal cavity wherein it may be expanded to adopt the operational configuration; the device comprising a flexible member coupled to an anchor element and wherein in an operational configuration the anchor element provides for an internal anchoring of the device at a predetermined position within the abdominal cavity and the flexible member is pivotable about the anchor element to contact with and lift a desired organ to a retracted position, and wherein the flexible member defines a web having a longitudinal axis extending substantially transverse to a longitudinal axis of the anchor, the flexible material having a length greater than its width, the web extending outwardly from the anchor in a direction parallel with its longitudinal axis.

2. (canceled)

3. The device of claim 1 wherein the flexible member is furled or rolled to adopt the insertion configuration.

4-5. (canceled)

6. The device of claim 1 wherein the anchor element is dimensioned to be receivable within a predetermined anatomical feature, where once inserted, the weight of one or more organs acting on the anchor will retain the anchor in situ providing for an internal anchoring of the device within the abdominal cavity.

7. The device of claim 1 wherein the diameter of the anchor element is less than 1.0 cm, to allow for its presentation of the anchor element through a trocar or cannula into the abdominal cavity

8-9. (canceled)

10. The device of claim 1 wherein the flexible member comprises a contact surface which operationally is in contact with the desired organ when implanted and flexes to conform to a contour of the organ when the contact surface is in contact with the desired organ.

11. (canceled)

12. The device of claim 10 wherein the contact surface is textured or treated to increase a coefficient of friction of the contact surface so as to increase adhesion between the device and the desired organ.

13. The device of claim 12 wherein the contact surface comprises a plurality of mechanical anti-slip elements or an adhesive to provide a temporary and controlled peak adhesive bond between the contact surface and the desired organ.

14. (canceled)

15. The device of claim 1 wherein on deployment of the device to provide for assisted lift of a desired organ, the flexible member is configured to be coupled to a suture which may be passed internally from the abdominal cavity out through the abdominal wall and secured externally via a tether placed on an outer surface of the abdominal wall or other portion of the torso.

16. The device of claim 1 wherein on deployment of the device to provide for assisted lift of an desired organ, the device is configured to be secured against an inner surface of the abdominal wall using a securing member or an adhesive coating to bond at least a portion of the retraction device to an inner surface of the abdominal wall to secure a deployed device in location.

17. (canceled)

18. The device of claim 1 wherein the anchor is dimensioned to be receivable within a fissure provided between a left lobe and a caudate lobe of a liver.

19. (canceled)

20. The device of claim 1, further comprising a plurality of anchors individually coupled to the flexible member.

21. The device of claim 1 wherein the flexible member is dimensioned to envelop a lower portion of an organ at two sides thereof so as to operably effect a lift of the organ out of a field of view of a surgeon.

22-25. (canceled)

26. The device of claim 1 wherein the flexible member comprises a plurality of apertures provided in a surface thereof, the apertures operably allowing organ tissue to invaginate into the material forming the flexible member to provide for improved grip.

27. The device of claim 1 wherein the flexible member comprises at least one strap portion moveable relative to a main body portion of the flexible member, the strap portion being operably tightened about a retracted organ to secure the organ within the flexible member.

28. (canceled)

29. The device of claim 1 comprising a shape memory material.

30. The device of claim 29 wherein the shape memory material is provided as contacting the flexible member which on adoption of the operational configuration biases the flexible member to adopt an expanded configuration.

31. The device of claim 30 wherein the shape memory material comprises a plurality of distinct elements arranged as ribs within the flexible member.

32. The device of claim 29 wherein the anchor element is fabricated from the shape memory material.

33. (canceled)

34. The device of claim 1 further comprising a drawstring provided in co-operation with the flexible member, the drawing coupled such that an application of tension to the drawstring effecting a corresponding change in a shape of the flexible member.

35-40. (canceled)

41. The device of claim 1 wherein at least a portion of the flexible member is receivable into an interior volume of the anchor element.

42. The device of claim 42 wherein the anchor element is configured to provide a controlled release of the flexible member from the interior volume.

43. The device of claim 1 wherein the anchor element comprises first and second portions which are pivotable relative to one another.

44-45. (canceled)

46. The device of claim 1 wherein the flexible member comprises a rib provided about a perimeter of the flexible member.

47-50. (canceled)

51. The method of claim 53 wherein positioning the anchor element of the device relative to the organ includes positioning the anchor element of the device relative to one of a small or a large bowel during a small or a large Bowel procedure.

52. (canceled)

53. A method of retracting an organ to allow surgical access to a surgical site during laparoscopic surgery, the method comprising:
providing a laparoscopic surgical retraction device comprising a flexible material coupled to an anchor element, the device having an insertion configuration and an operational configuration wherein in the insertion configuration the device is collapsible such that the dimensions of the device can be reduced so as to allow the complete insertion of the device through a trocar or cannula into an internal abdominal cavity wherein it may be expanded to adopt the operational configuration;
inserting the retraction device fully into the abdominal cavity through a trocar or cannula;
expanding the flexible member of the device to allow adoption of the operational configuration;
positioning the anchor element of the device relative to the organ to secure the anchor relative to the organ;
contacting the organ with the flexible member and pivoting the flexible member relative to the secured anchor to effect a moving of the organ and
securing the organ in situ by securing the device relative to the abdominal wall.

54. The method of claim 53 wherein the securing of the anchor is effected through disposing the anchor element under the organ and using the weight of the organ onto the anchor element.
